(19) United States
(12) Patent Application Publication
(10) Pub. No.: US 2015/0320378 A1
(43) Pub. Date: Nov. 12, 2015

(54) X-RAY DIAGNOSIS APPARATUS

(71) Applicants: Kabushiki Kaisha Toshiba, Minato-ku (JP); Toshiba Medical Systems Corporation, Otawara-shi (JP)

(72) Inventors: Manabu TANAKA, Otawara (JP); Yoshinori SHIMIZU, Nasushiobara (JP); Takayuki ISHIKAWA, Nasushiobara (JP); Teruomi GUNJI, Otawara (JP)

(73) Assignees: Kabushiki Kaisha Toshiba, Minato-ku (JP); Toshiba Medical Systems Corporation, Otawara-shi (JP)

(21) Appl. No.: 14/807,119

(22) Filed: Jul. 23, 2015

Related U.S. Application Data
(63) Continuation of application No. PCT/JP2014/051275, filed on Jan. 22, 2014.

(30) Foreign Application Priority Data
Jan. 23, 2013 (JP) 2013-009819
Jan. 22, 2014 (JP) 2014-009046

(51) Int. Cl. A61B 6/00; A61B 6/04

(52) U.S. Cl. CPC A61B 6/464; A61B 6/0407; A61B 6/4441; A61B 6/0457

(57) ABSTRACT

According to one embodiment, an X-ray diagnosis apparatus includes a couch where a subject is placed, a projector, a display controller, and a system controller. The projector includes an X-ray tube having a cathode and an anode that receives electrons from the cathode and irradiates X-rays to the subject, and a first detector configured to detect X-rays that have passed through the subject and are incident on the detection surface. The display controller displays a first image generated based on first detection data from the projector on a display. Having received an enlargement instruction to display an enlarged image of part of the subject illustrated in the first image, the system controller controls the display controller to display a second image generated based on second detection data obtained by detecting X-rays incident on the partial detection surface corresponding to the anode side in the detection surface as the enlarged image.

### CROSS-REFERENCE TO RELATED APPLICATION

This application is based upon and claims the benefit of priority from Japanese Patent Applications No. 2013-009819, filed 23 Jan. 2013, and No. 2014-009046, filed 22 Jan. 2014; the entire contents of which are incorporated herein by reference.

### FIELD

Embodiments described herein relate generally to an X-ray diagnosis apparatus.

### BACKGROUND

An X-ray diagnosis apparatus irradiates a subject with X-rays and detects the X-rays having passed therethrough, thereby imaging the internal structure of the subject.

There has been a known X-ray diagnosis apparatus that includes a C-shaped supporting device (C-arm), an X-ray tube arranged at one end of the supporting device, a flat panel detector arranged at the other end of the supporting device, a couch on which the subject is placed, and an image processing unit that processes projection data collected.

The X-ray diagnosis apparatus takes X-rays in parallel to doctor's work, such as the insertion of a catheter into a subject in surgery or examination (diagnosis). At this time, the doctor performs surgery or examination while viewing captured images to acquire the internal structure of the subject.

On occasion, there may be a case where the X-ray diagnosis apparatus receives an instruction to display an enlarged image. If this happens, the X-ray diagnosis apparatus converts the enlarged size specified by the instruction to an area in the detection surface of the flat panel detector, and assigns the area to the flat panel detector such that the center of the visual field is fixed in the detection surface. Then, the X-ray diagnosis apparatus displays an image generated from X-rays incident on the region.

The X-ray tube includes a cathode and an anode. Electrons from the cathode collide with the anode, and thus the anode generates X-rays to be irradiated to the subject. The X-rays are emitted with a spread angle.

The flat panel detector detects X-rays that have passed through the subject and are incident on the detection surface. The X-ray diagnosis apparatus generates an image based on the incident X-rays and displays it. It is known that, of these images, an image based on the projection data of X-rays incident from the anode side of the spread angle has a better resolution than the image based on the projection data of X-rays incident from the cathode side. In other words, the resolution of images is known to be reduced from the anode side to the cathode side.

A conventional X-ray diagnosis apparatus displays an enlarged image such that the center of the visual field is fixed in the detection surface of the flat panel detector. Therefore, a portion with a reduced resolution in an image before being enlarged is displayed in an enlarged scale. Accordingly, the doctor is necessitated to see the enlarged image of poor resolution, which lowers the visibility of the enlarged image by the doctor. A reduction in the visibility of the image may lead to prolonged surgery or examination, resulting in an increase in the radiation exposure of the subject due to the prolonged time.

### BRIEF DESCRIPTION OF THE DRAWINGS

FIG. 1 is a block diagram of an X-ray diagnosis apparatus according to an embodiment;

FIG. 2 is a schematic diagram of the X-ray diagnosis apparatus of the embodiment;

FIG. 3 is a schematic diagram of an X-ray tube of the embodiment;

FIG. 4 is a schematic diagram of a projector of the embodiment;

FIG. 5A is a schematic diagram of a detection surface of the embodiment;

FIG. 5B is a schematic diagram of the detection surface of the embodiment;

FIG. 6 is a schematic diagram of a first isocenter and a second isocenter of the embodiment;

FIG. 7 is a flowchart of the operation of the X-ray diagnosis apparatus of the embodiment;

FIG. 8A is a schematic diagram of a couch, a subject, and a projector according to another embodiment;

FIG. 8B is a schematic diagram of the couch, the subject, and the projector of the embodiment;

FIG. 8C is a schematic diagram of the couch, the subject, and the projector of the embodiment;

FIG. 9 is a flowchart of the operation of the X-ray diagnosis apparatus of the embodiment;

FIG. 10 is a block diagram of an X-ray diagnosis apparatus according to an embodiment;

FIG. 11A is a schematic diagram of a projector of the embodiment;

FIG. 11B is a schematic diagram of the projector of the embodiment;

FIG. 12A is a schematic diagram of a couch, a subject, and the projector of the embodiment;

FIG. 12B is a schematic diagram of the couch, the subject, and the projector of the embodiment;

FIG. 12C is a schematic diagram of the couch, the subject, and the projector of the embodiment; and

FIG. 13 is a flowchart of the operation of the X-ray diagnosis apparatus of the embodiment.

### DETAILED DESCRIPTION

In general, according to one embodiment, an X-ray diagnosis apparatus includes a couch on which a subject is placed, a projector, a display controller, and a system controller. The projector includes an X-ray tube having a cathode and an anode that receives electrons from the cathode and irradiates X-rays to the subject, and a first detector configured to detect X-rays that have passed through the subject and are incident on the detection surface. The display controller displays a first image generated based on first detection data from the projector on a display. Having received an enlargement instruction to display an enlarged image of part of a site of the subject illustrated in the first image, the system controller controls the display controller to display a second image generated based on second detection data obtained by detecting X-rays incident on the partial detection surface that is a partial area corresponding to the anode side in the detection surface as the enlarged image on the display.

### First Embodiment

Referring now to the drawings, a description is given of an X-ray diagnosis apparatus according to embodiments.

### [Basic Configuration]

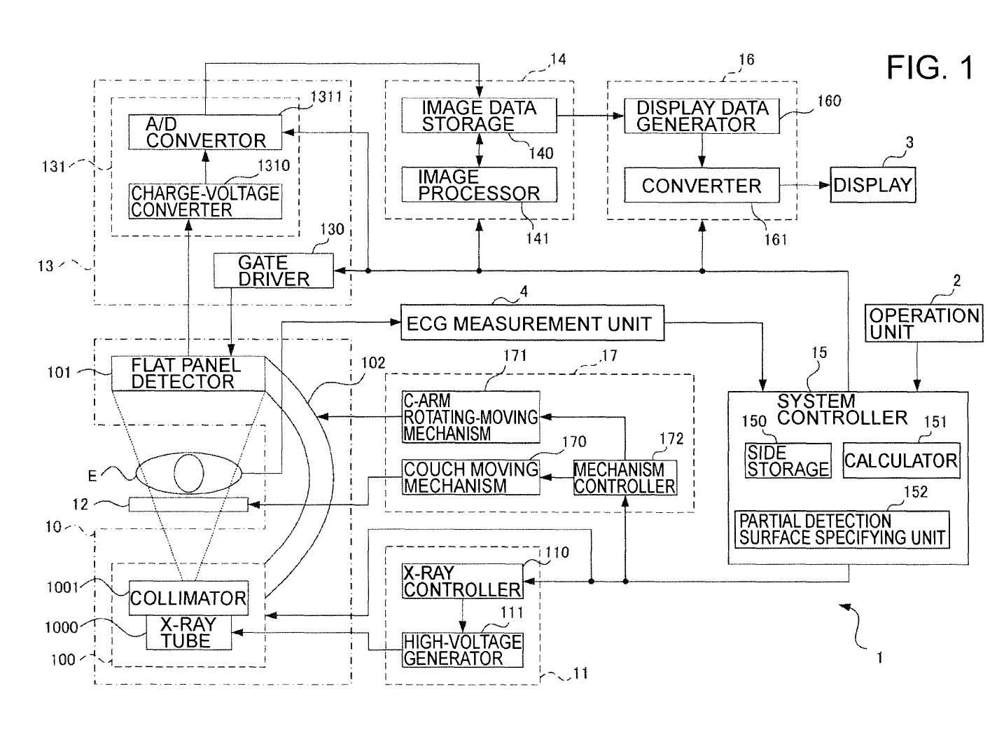

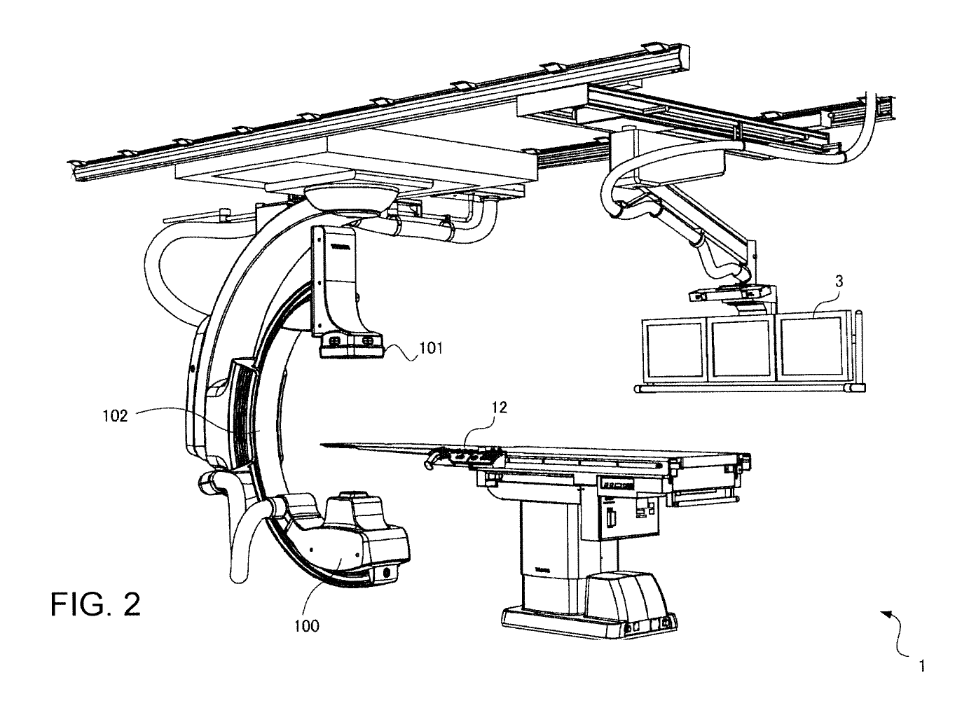

FIG. 1 is a block diagram illustrating a configuration of an X-ray diagnosis apparatus 1 of the embodiment. FIG. 2 is a schematic diagram illustrating an appearance of the X-ray diagnosis apparatus 1. The X-ray diagnosis apparatus 1 includes a projector 10, a high-voltage generating unit 11, a couch 12, an X-ray detector 13, an image data generator 14, a system controller 15, an operation unit 2, a display controller 16, a display 3, an ECG measurement unit 4, and a mechanism 17.

### (Projector 10)

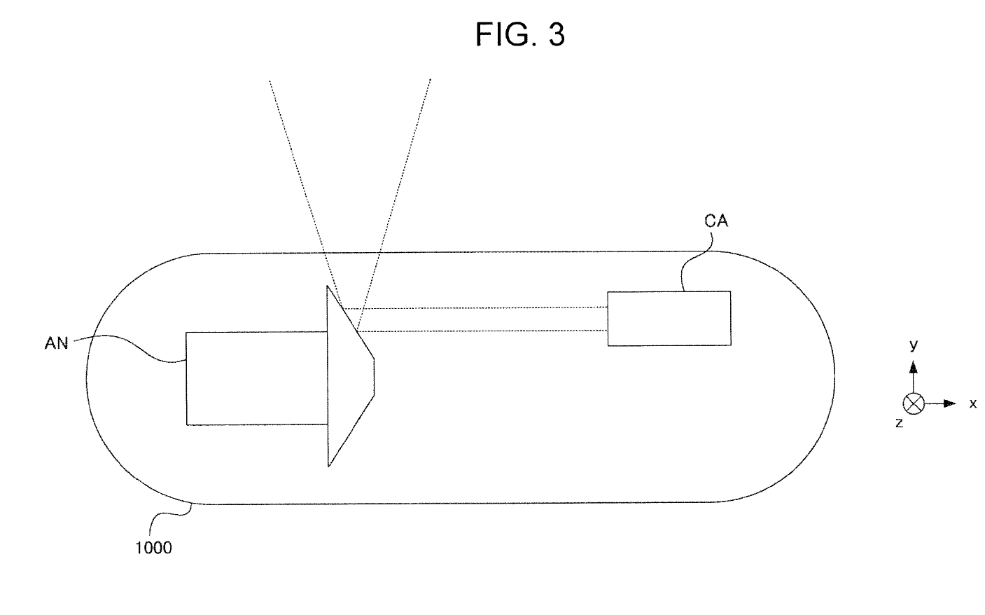

The projector 10 includes an X-ray generator 100, a flat panel detector 101, and a C-arm 102. The X-ray generator 100 irradiates X-rays to a subject E. The X-ray generator 100 includes an X-ray tube 1000 and an collimator 1001. The X-ray tube 1000 is a vacuum tube that generates X-rays. FIG. 3 is a schematic diagram illustrating the outline of the X-ray tube 1000. The X-ray tube 1000 includes a cathode CA and an anode AN. The cathode CA emits electrons. The electrons from the cathode collide with the anode AN, and thus the anode AN generates X-rays, and irradiates them to the subject E. Here, the negative side of the x axis corresponds to the anode AN side, and the positive side corresponds to the cathode CA side. As described above, images based on X-rays on the anode AN side have a better resolution than those based on X-rays on the cathode CA side. The collimator 1001 is located between the X-ray tube 1000 and the subject E. The collimator 1001 forms a slit (opening), and adjusts the irradiation field of X-rays generated by the X-ray tube 1000 by changing the size and shape of the slit.

The flat panel detector 101 detects X-rays that have passed through the subject E and are incident on the detection surface. For example, the flat panel detector 101 converts the X-rays incident on the detection surface into electric charge and accumulates it. The flat panel detector 101 includes two-dimensional arrays of a plurality of X-ray detection elements on the detection surface. The X-ray detection element is provided with a photoelectric film, a charge storage capacitor, and a thin film transistor (TFT). The photoelectric film detects X-rays, and generates electric charge according to the dose of the X-rays detected. The charge storage capacitor stores the charge generated by the photoelectric film. The TFT retrieves the charge accumulated in the charge storage capacitor. The flat panel detector 101 outputs the charge retrieved by the TFT to a charge-voltage converter 1310 as detection data. The flat panel detector 101 corresponds to an example of “first detector”.

The C-arm 102 supports the X-ray generator 100 and the flat panel detector 101. The C-arm 102 has a C shape, and is provided with the X-ray tube 1000 at one end and the flat panel detector 101 at the other end.

### (High-Voltage Generating Unit 11)

The high-voltage generating unit 11 generates a high voltage for the X-ray generator 100 to irradiate X-rays. The high-voltage generating unit 11 includes an X-ray controller 110 and a high-voltage generator 111. The X-ray controller 110 outputs a control signal regarding X-ray irradiation conditions, such as tube current and tube voltage of the X-ray tube 1000, irradiation time, and the like, to the high-voltage generator 111 based on input from the system controller 15. The high-voltage generator 111 applies a high voltage between the anode AN and the cathode CA of the X-ray tube 1000 based on the input from the X-ray controller 110.

### (Couch 12)

On the couch 12 is placed the subject E. The couch 12 moves the subject E placed thereon in its body axis direction and the vertical direction based on input from a couch moving mechanism 170.

### (X-Ray Detector 13)

The X-ray detector 13 includes a gate driver 130 and a projection data generator 131. The gate driver 130 outputs a drive pulse for reading out to the gate terminal of the TFT to allow the TFT to retrieve the charge stored in the charge storage capacitor.

The projection data generator 131 generates projection data based on the detection data from the flat panel detector 101. The projection data generator 131 includes the charge-voltage converter 1310 and an A/D convertor 1311. The charge-voltage converter 1310 converts the electric charge received as the detection data from the flat panel detector 101 into a voltage, and outputs a signal of the voltage to the A/D convertor 1311. Upon receipt of the signal from the charge-voltage converter 1310, the A/D converter 1311 converts it into a digital signal. The A/D converter 1311 outputs the digital signal to an image data storage 140.

### (Image Data Generator 14)

The image data generator 14 generates image data representing the internal structure of the subject E based on the projection data generated by the projection data generator 131 and stores it. The image data generator 14 includes the image data storage 140 and an image processor 141. The image data storage 140 stores the projection data from the projection data generator 131 and the image data from the image processor 141. The image data storage 140 outputs the projection data to the image processor 141. The image data storage 140 outputs the image data to a display data generator 160.

Having received the projection data from the image data storage 140, the image processor 141 performs various types of image processing on the projection data, thereby generating the image data representing the internal structure of the subject E. The image processor 141 outputs the image data thus generated to the image data storage 140.

### (System Controller 15)

The system controller 15 is an example of second circuitry in claims. The system controller 15 once stores information such as a command signal and shooting conditions provided by the user through the operation unit 2, and thereafter, controls each unit such as the mechanism 17 for the generation of X-ray projection data based on the information, the generation and display of the image data, or the like. The system controller 15 includes, for example, a processing unit and a storage device. Examples of the processing unit include, for example, a central processing unit (CPU), a graphics processing unit (GPU), and an application specific integrated circuit (ASIC). Examples of the storage device include a read only memory (ROM), a random access memory (RAM), and a hard disc drive (HDD). The storage device stores a computer program for implementing the functions of each unit of the X-ray diagnosis apparatus 1. The processing unit executes the computer programs to implement the above functions.

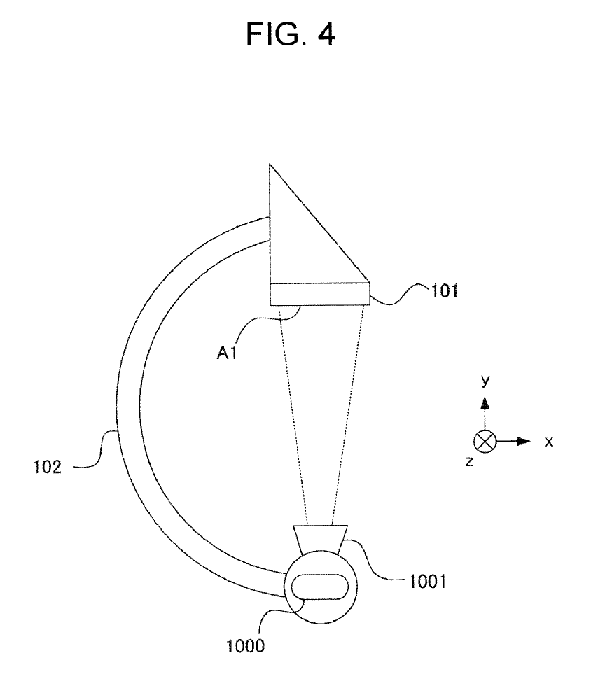

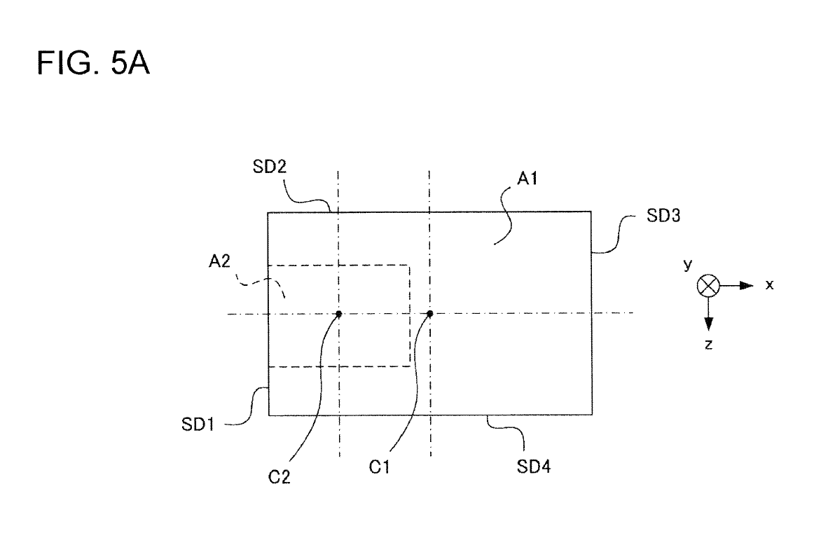

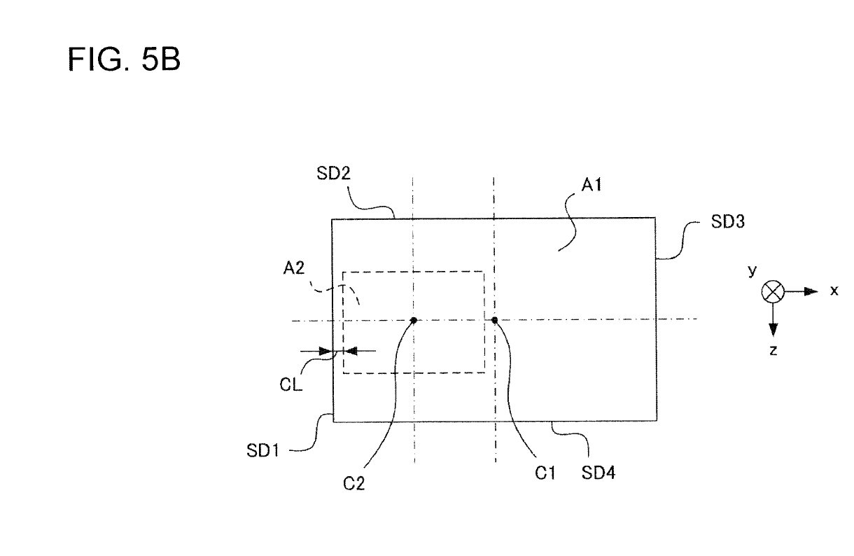

The system controller 15 includes a side storage 150, a calculator 151, and a partial detection surface specifying unit 152. The side storage 150 stores in advance a side corresponding to the anode AN side of the X-ray tube 1000 from among the sides of the detection surface of the flat panel detector 101. The side is used as a reference for a partial detection surface (described later). FIG. 4 is a schematic diagram illustrating the relationship between the flat panel detector 101 and the X-ray tube 1000 in the projector 10. In this embodiment, the anode AN of the X-ray tube 1000 is located on the −x side, while the cathode CA is located on the +x side. That is, an image based on X-rays on the −x side has a better resolution than an image based on X-rays on the +x side. FIGS. 5A and 5B are schematic diagrams illustrating a detection surface A1 of the flat panel detector 101 of FIG. 4 viewed from the −y side toward the direction of +y side and the relationship between the detection surface A1 and a partial detection surface A2. The detection surface A1 has sides SD1, SD2, SD3, and SD4. In this embodiment, the side SD1 located on the −x side corresponds to the anode AN side. The side corresponding to the anode AN side is determined by the positional relationship between the anode AN and the cathode CA of the X-ray tube 1000 in the projector 10. This positional relationship may be appropriately designed depending on the type of the X-ray diagnosis apparatus 1. For example, in models with the X-ray tube 1000 having the anode AN on the −z side and the cathode CA on the +z side, the side SD2 corresponds to the anode AN side. Similarly, in models with the X-ray tube 1000 having the anode AN on the +x side and the cathode CA on the −x side, the side SD3 corresponds to the anode AN side. In addition, in models with the X-ray tube 1000 having the anode AN on the +z side and the cathode CA on the −z side, the side SD4 corresponds to the anode AN side. In this embodiment, an example is described in which the side SD1 is a side corresponding to the anode AN side. The partial detection surface A2 is described later.

### (Operation Unit 2)

When operated by the user, the operation unit 2 feeds a signal or information corresponding to the operation to each unit. The operation unit 2 may include, for example, a keyboard, a mouse, and various types of switches.

### (Display Controller 16)

The display controller 16 is an example of first circuitry in claims. The display controller 16 displays a first image generated based on first detection data from the projector 10 on the display 3. Having received an enlargement instruction for enlarged display of a portion of the first image, the display controller 16 provides, on the display 3, enlarged display of a second image generated based on second detection data obtained by detecting X-rays incident on a partial detection surface that is a partial area corresponding to the anode AN side in the detection surface A1. For example, based on a side corresponding to the anode AN side in the sides of the detection surface A1 and the enlarged size contained in the enlargement instruction, the display controller 16 provides, on the display 3, enlarged display of the second image generated based on the second detection data obtained by detecting X-rays incident on the partial detection surface A2, which is a partial area of the detection surface A1 having at least a part of the side corresponding to the anode AN side as a side.

The display controller 16 includes the display data generator 160 and a converter 161. Having received the image data from the image data storage 140, the display data generator 160 adds desired text information or the like to the image data as additional information to generate display data. Having received the display data from the display data generator 160, the converter 161 performs D/A conversion and TV format conversion on the display data to generate a video signal. The converter 161 outputs the video signal to the display 3. The display controller 16 displays an image on the display 3.

### (Display 3)

The display 3 receives the display data from the display controller 16 and displays an image. The display 3 may be formed of a display device such as, for example, a liquid crystal display (LCD), a cathode ray tube (CRT), or the like.

### (ECG Measurement Unit 4)

The ECG measurement unit 4 measures electrocardiogram (ECG) of the subject E, and outputs the ECG to the system controller 15. Incidentally, the ECG measurement unit 4 is not necessarily a part of the X-ray diagnosis apparatus 1, and may be connected from the outside to the X-ray diagnosis apparatus 1 via a general interface.

### (Mechanism 17)

The mechanism 17 causes relative movement between the couch 12 and the projector 10. The mechanism 17 includes the couch moving mechanism 170, a C-arm rotating-moving mechanism 171, and a mechanism controller 172. The couch moving mechanism 170 moves the couch 12 in the body axis direction of the subject E and a direction perpendicular to the body axis direction. The C-arm rotating-moving mechanism 171 rotates the C-arm 102 around the subject E, and also moves the C-arm 102 in parallel. The mechanism controller 172 controls the couch moving mechanism 170 and the C-arm rotating-moving mechanism 171 based on input from the system controller 15.

### [Enlarged Display]

A description is given of the configuration of the X-ray diagnosis apparatus 1 for displaying an enlarged image of part of a site of the subject illustrated in an image. Here, the image before enlargement is referred to as “first image”, and the image displayed in response to an enlargement instruction is referred to as “second image”.

Upon receipt of an enlargement instruction, the system controller 15 controls the display controller 16 so that the display 3 displays, as an enlarged image, a second image generated based on second detection data obtained from X-rays incident on a partial detection surface that is a partial area corresponding to the anode AN side in the detection surface A1. For example, the system controller 15 outputs enlarged size contained in the enlargement instruction and a side corresponding to the anode AN side stored in the side storage 150 in advance to the partial detection surface specifying unit 152. The enlargement instruction is, for example, provided by the user through the operation unit 2 to the system controller 15.

The system controller 15 controls the partial detection surface specifying unit 152 to specify the partial detection surface A2. The center C2 of the partial detection surface A2 is only required to be on the anode AN side compared to the center C1 of the detection surface A1. The partial detection surface specifying unit 152 specifies, as the partial detection surface A2, an area of the detection surface A1 having at least part of the side SD1 corresponding to the anode AN side as a side based on the enlarged size. For example, when the enlarged size is twice as large in vertical and horizontal dimensions of an image (the area is 4 times larger), the partial detection surface A2 is an area having vertical and horizontal lengths half of those of the detection surface A1 with a part of the SD1 as a side (see FIG. 5A). The partial detection surface specifying unit 152 may specify, as the partial detection surface A2, a part of the detection surface A1 with the line segment located at the distance CL from the side SD1 as a side (see FIG. 5B). In other words, the partial detection surface specifying unit 152 may specify an area having a side located at the distance CL from the side SD1 as the partial detection surface A2. Here, the distance CL may be preset, for example, or may be specified by the user. The partial detection surface specifying unit 152 may specify the partial detection surface A2 such that the position in a direction parallel to the side SD1 (z-axis coordinates in FIGS. 5A and 5B) matches between the center C1 of the detection surface A1 and the center C2 of the partial detection surface A2.

Having been informed of the partial detection surface A2 from the system controller 15, the display controller 16 provides, on the display 3, enlarged display of a second image generated based on the second detection data obtained by detecting X-rays incident on the partial detection surface A2.

When providing the enlarged display of the second image on the display 3, for example, the display controller 16 may read the coordinates of the image data received from the image data generator 14, and generate the display data from part of the image data for an area contained in the partial detection surface A2. The display controller 16 outputs the display data to the display 3 to provide an enlarged display as the second image on the display 3.

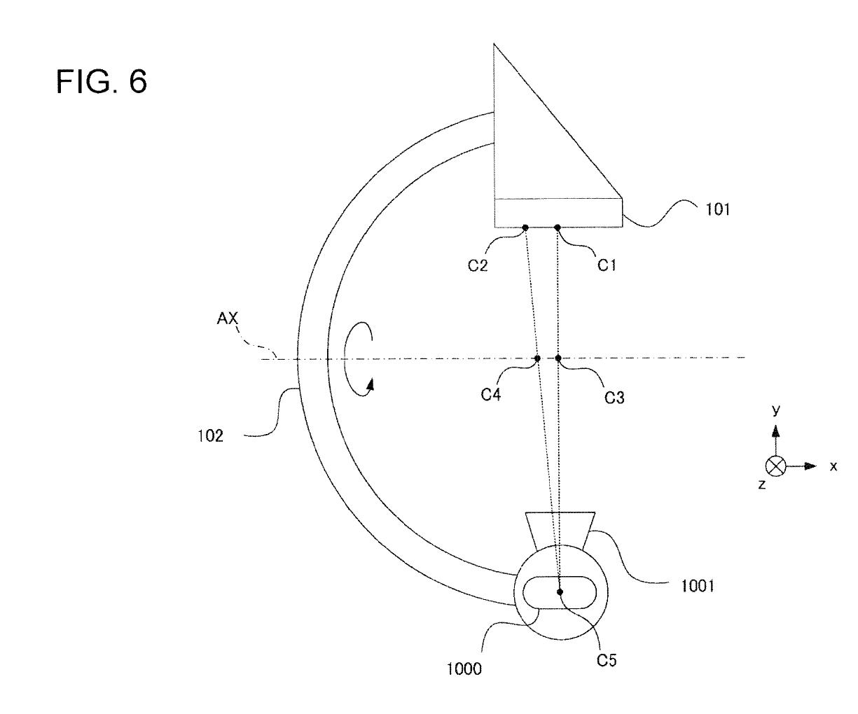

Incidentally, upon receipt of an enlargement instruction, the system controller 15 may output, to the mechanism 17, a displacement instruction representing a direction in which the second isocenter corresponding to the partial detection surface A2 is brought close to the position of the first isocenter corresponding to the detection surface A1 based on the detection surface A1 and the partial detection surface A2. For example, the calculator 151 calculates the displacement amount of the second isocenter relative to the first isocenter. FIG. 6 is a schematic diagram illustrating the positional relationship between a first isocenter C3 and a second isocenter C4. The first isocenter C3 is an intersection between a straight line connecting the center C2 of the detection surface A1 and an X-ray focal point C5 and the rotation axis AX of the C-arm 102. The second isocenter C4 is an intersection between a straight line connecting the center of the partial detection surface A2 and the X-ray focal point C5 and the rotation axis AX of the C-arm 102. Here, the distance between the X-ray focal point C5 and the center C1 of the detection surface A1, and the distance between the X-ray focal point C5 and the first isocenter C3 are known. Besides, when the partial detection surface A2 is specified, the distance between the center C1 of the detection surface A1 and the center C2 of the partial detection surface A2 is known. The calculator 151 calculates the displacement amount of the second isocenter C4 with respect to the first isocenter C3 based on the similarity relationship between a triangle formed of the X-ray focal point C5, the center C1 of the detection surface A1, and the center C2 of the partial detection surface A2 and a triangle formed of the X-ray focal point C5, the first isocenter C3, and the second isocenter C4. The calculator 151 outputs the displacement amount thus obtained to the system controller 15. The system controller 15 outputs a displacement instruction corresponding to the displacement amount received from the calculator 151 to the mechanism 17. The displacement instruction refers to an instruction to control the mechanism 17 to relatively move the couch 12 and the projector 10 so that the position of the second isocenter C4 matches the position of the first isocenter C3 before enlargement instruction.

Upon receipt of the displacement instruction from the system controller 15, the mechanism 17 relatively moves the couch 12 and the projector 10 to match the position of the first isocenter C3 with the position of the second isocenter C4. Here, the mechanism controller 172 of the mechanism 17 controls the C-arm rotating-moving mechanism 171 based on the displacement instruction to move the projector 10 in a direction in which the position of the first isocenter C3 and the position of the second isocenter C4 get close to each other, that is, in a direction from the second isocenter C4 to the first isocenter C3, thereby matching the position of the second isocenter C4 with the position of the first isocenter C3. Note that the matching accuracy at this time is a design matter of the X-ray diagnosis apparatus 1 and may be designed within the range of errors tolerable in practical use. Further, the mechanism controller 172 may control the couch moving mechanism 170 to move the couch 12 in a direction in which the position of the first isocenter C3 and the position of the second isocenter C4 get close to each other, that is, in a direction from the first isocenter C3 to the second isocenter C4, thereby matching the position of the second isocenter C4 with the position of the first isocenter C3. Still further, the mechanism controller 172 may control both the couch moving mechanism 170 and the C-arm rotating-moving mechanism 171 to move both the couch 12 and the projector 10 to thereby match the position of the second isocenter C4 with the position of the first isocenter C3. Thus, the center of the first image and the center of the second image match, and the display 3 displays an enlarged image of a desired position.

Incidentally, upon receipt of an enlargement instruction, the system controller 15 may control the collimator 1001 based on a side corresponding to the anode AN side and the enlarged size contained in the enlargement instruction to form a slit for irradiating the partial detection surface A2 with X-rays. In this case, the size of the slit is reduced compared to before the receipt of the enlargement instruction, and the position of the slit is biased in the direction of the side corresponding to the anode AN side.

### [Operation]

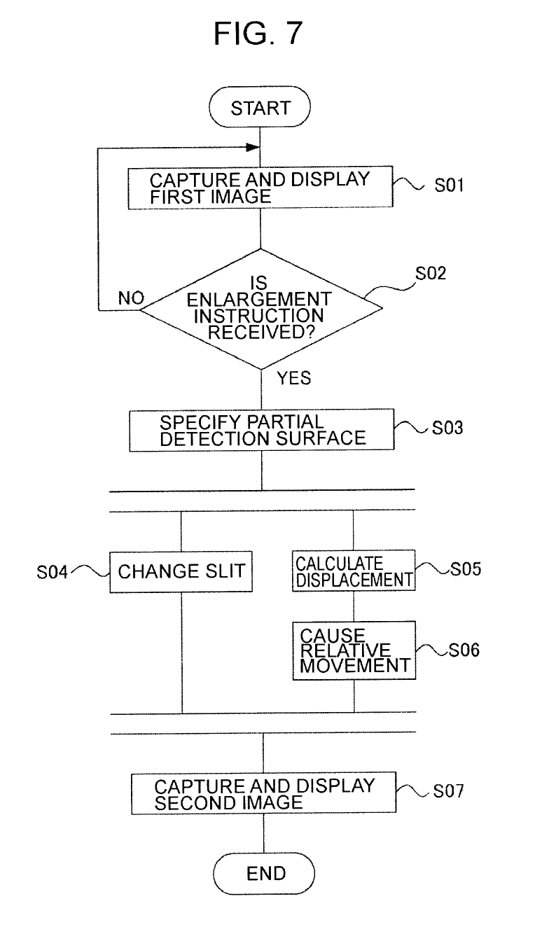

Described below is the operation of the X-ray diagnosis apparatus 1. FIG. 7 is a flowchart illustrating the operation of the X-ray diagnosis apparatus 1.

### (Step S01)

The X-ray diagnosis apparatus 1 captures the first image and displays it on the display 3. At this time, the X-ray generator 100 irradiates the detection surface A1 with X-rays, and the flat panel detector 101 detects X-rays that have passed through the subject E and are incident on the detection surface A1. The flat panel detector 101 outputs first detection data to the projection data generator 131. The projection data generator 131 outputs projection data based on the first detection data to the image data generator 14. The image data generator 14 generates image data based on the projection data, and outputs it to the display controller 16. The display controller 16 generates display data based on the image data, and displays it on the display 3.

### (Steps S02, S03)

Having received an enlargement instruction (YES in step S02), the system controller 15 outputs the enlarged size contained in the enlargement instruction and a side corresponding to the anode AN side stored in the side storage 150 to the partial detection surface specifying unit 152. The partial detection surface specifying unit 152 outputs the partial detection surface A2 specified to the calculator 151 and the collimator 1001. On the other hand, when the system controller 15 receives no enlargement instruction (NO in step S02), the process returns to step S01.

### (Step S04)

The collimator 1001 changes the slit based on the partial detection surface A2 fed from the partial detection surface specifying unit 152. Thus, X-rays are irradiated from the X-ray tube 1000 toward the partial detection surface A2.

### (Step S05)

The calculator 151 calculates the displacement amount of the second isocenter C4 corresponding to the partial detection surface A2 with respect to the first isocenter C3 corresponding to the detection surface A1 based on the detection surface A1 and the partial detection surface A2 fed from the partial detection surface specifying unit 152. The calculator 151 outputs the displacement amount thus obtained to the system controller 15. The system controller 15 outputs a displacement instruction corresponding to the displacement amount fed from the calculator 151 to the mechanism 17.

### (Step S06)

Having received the displacement instruction from the system controller 15, the mechanism 17 relatively moves the couch 12 and the projector 10 to match the position of the second isocenter C4 with the position of the first isocenter C3. Incidentally, the steps S04, S05, and S06 are in the parallel processing relationship.

### (Step S07)

The X-ray diagnosis apparatus 1 captures the second image and displays it on the display 3. At this time, the X-ray generator 100 irradiates the partial detection surface A2 with X-rays, and the flat panel detector 101 detects X-rays that have passed through the subject E and are incident on the partial detection surface A2. The flat panel detector 101 outputs second detection data to the projection data generator 131. The projection data generator 131 outputs projection data based on the second detection data to the image data generator 14. The image data generator 14 generates image data based on the projection data, and outputs it to the display controller 16. The display controller 16 generates display data based on the image data to provide enlarged display on the display 3. With this, the operation of FIG. 7 ends.

According to this embodiment, the X-ray diagnosis apparatus 1 includes the couch 12 on which the subject E is placed, the projector 10, the display controller 16, and the system controller 15. The projector 10 includes the X-ray tube 1000 including a cathode CA and an anode AN that receives electrons from the cathode CA and irradiates X-rays to the subject E, and a first detector configured to detect X-rays that have passed through the subject E and are incident on the detection surface A1. The display controller 16 displays a first image generated based on first detection data from the projector 10 on the display 3. Having received an enlargement instruction to display an enlarged image of part of a site of the subject E illustrated in the first image, the system controller 15 controls the display controller 16 to display a second image generated based on second detection data obtained by detecting X-rays incident on the partial detection surface A2 that is a partial area corresponding to the anode AN side in the detection surface A1 as an enlarged image on the display 3. In this manner, upon receipt of an enlargement instruction for an image, the X-ray diagnosis apparatus 1 provides enlarged display of an image based on X-rays incident on the partial detection surface A2. Besides, X-rays on the anode AN side in the X-ray tube 1000 are incident on the partial detection surface A2, and an image based on the X-rays on the anode AN side has a good resolution. Thus, it is possible to improve the resolution of an enlarged image.

The system controller 15 includes the calculator 151. Having received an enlargement instruction, the calculator 151 calculates the displacement amount of the second isocenter C4 corresponding to the partial detection surface A2 with respect to the first isocenter C3 corresponding to the detection surface A1 based on the detection surface A1 and the partial detection surface A2, and outputs the displacement amount thus obtained to the system controller 15. The system controller 15 outputs a displacement instruction corresponding to the displacement amount fed from the calculator 151 to the mechanism 17. Upon receipt of the displacement instruction from the system controller 15, the mechanism 17 relatively moves the couch 12 and the projector 10 to match the position of the second isocenter C4 with the position of the first isocenter C3. In this manner, by matching the position of the first isocenter C3 before enlargement with the position of the second isocenter C4 after enlargement, a site of the subject E illustrated in the center of an image before being enlarged matches that of the subject E illustrated in the center of an enlarged image. Thus, the resolution of the enlarged image is improved. Further, it is possible to provide enlarged display of the same site while it is being displayed.

### Second Embodiment

### Configuration

Described below is an X-ray diagnosis apparatus of a second embodiment. This embodiment is different from the first embodiment in the configuration of the system controller 15 and the mechanism 17. In the following, the differences from the first embodiment are mainly explained.

Having received an enlargement instruction, the system controller 15 outputs, to the mechanism 17, a rotation instruction representing rotational movement that makes a second straight line connecting the center C2 of the partial detection surface A2 and the X-ray focal point C5 parallel to a first straight line connecting the center C1 of the detection surface A1 and the X-ray focal point C5.

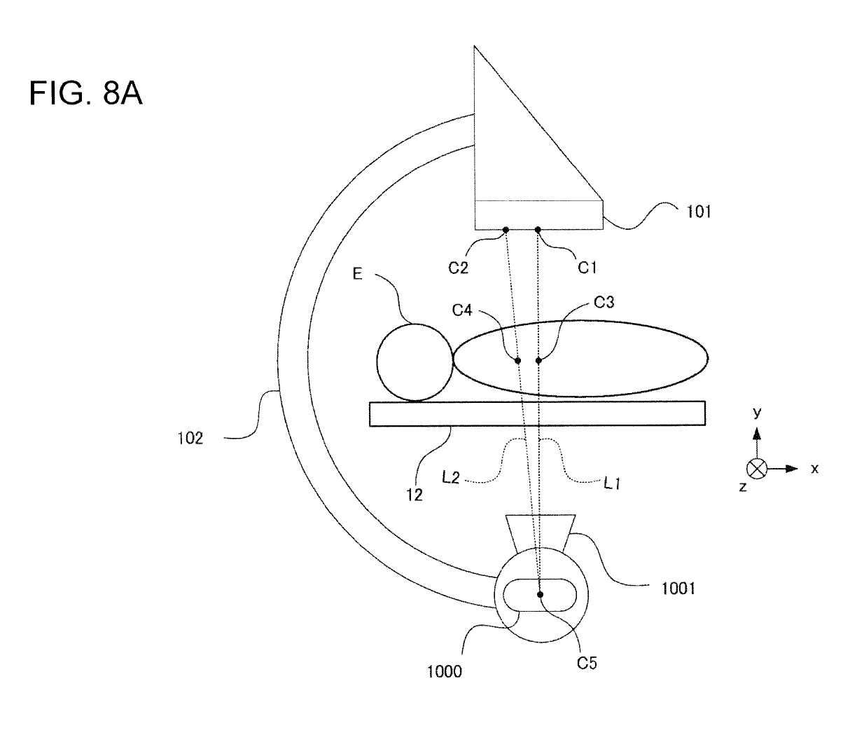

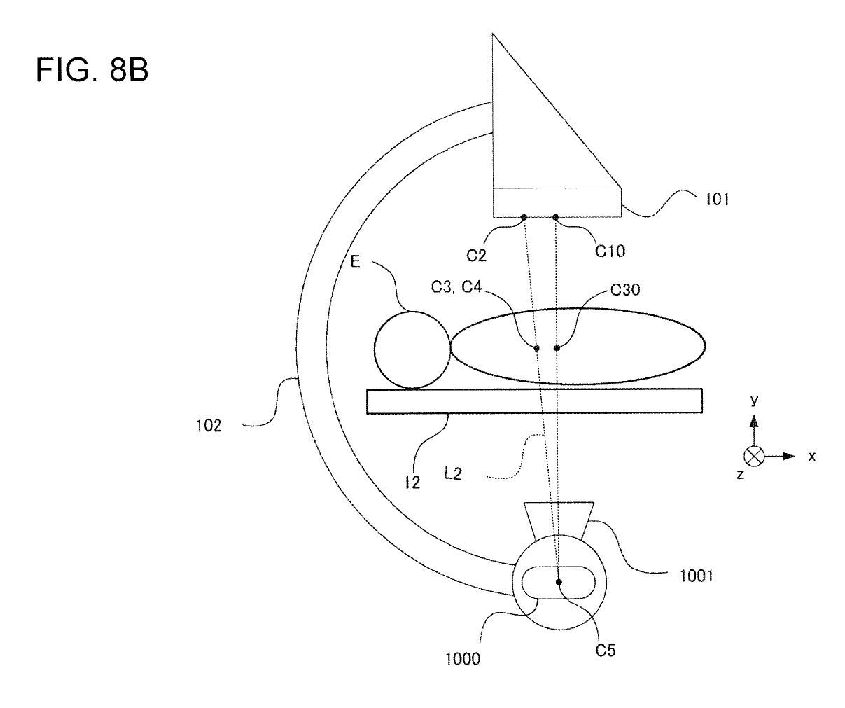

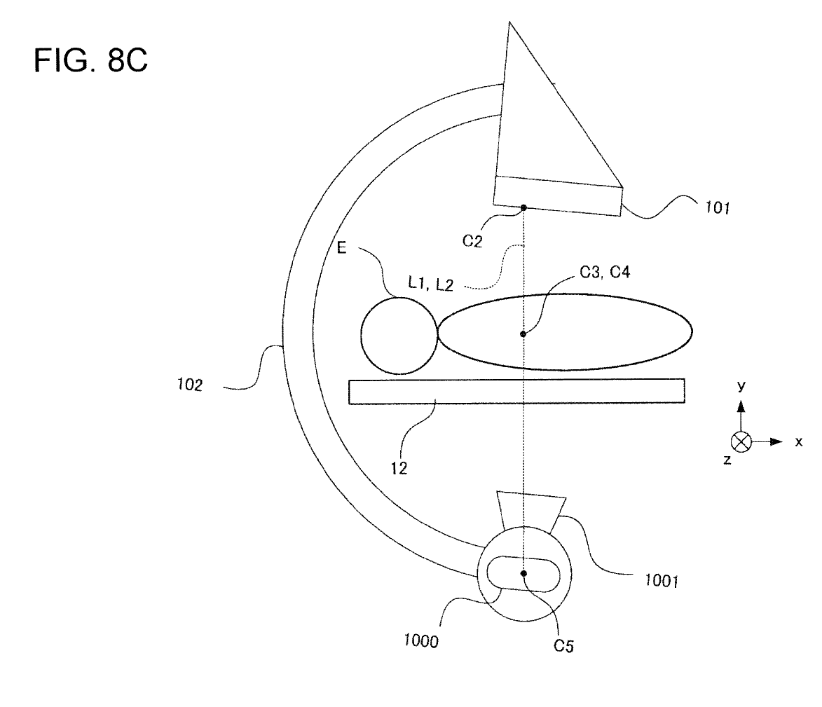

FIG. 8A is a schematic diagram illustrating the couch 12, the subject E, and the projector 10 before the mechanism 17 relatively moves the couch 12 and the projector 10. FIG. 8B is a schematic diagram illustrating the couch 12, the subject E, and the projector 10 when the mechanism 17 relatively moves the couch 12 and the projector 10. FIG. 8C is a schematic diagram illustrating the couch 12, the subject E, and the projector 10 when the mechanism 17 relatively rotates the couch 12 and the projector 10. When the mechanism 17 relatively moves the couch 12 and the projector 10, the direction of a first straight line L1 differs from the direction of a second straight line L2. The direction of the first straight line L1 corresponds to a direction in which X-rays are irradiated to the subject E before enlargement instruction is received. The direction of the second straight line L2 corresponds to a direction in which X-rays are irradiated to the subject E after an enlargement instruction is received.

For example, the system controller 15 calculates the angle between the direction of the first straight line L1 and the direction of the second straight line L2 based on the displacement amount obtained by the calculator 151. Based on the angle obtained, the system controller 15 outputs, to the mechanism 17, an instruction to relatively rotate the couch 12 and the projector 10, that is, a rotation instruction representing rotational movement that makes the first straight line L1 parallel to the second straight line L2.

Having received the rotation instruction, the mechanism 17 relatively rotates the couch 12 and the projector 10. At this time, the mechanism controller 172 of the mechanism 17 controls the C-arm rotating-moving mechanism 171 based on the rotation instruction to rotate the projector 10 to make the second straight line L2 parallel to the first straight line L1. Note that the parallel accuracy at the time is a design matter of the X-ray diagnosis apparatus and may be designed within the range of errors tolerable in practical use. Further, the mechanism controller 172 may control the couch moving mechanism 170 and rotate the couch 12 to make the second straight line L2 parallel to the first straight line L1. Still further, the mechanism controller 172 may control both the couch moving mechanism 170 and the C-arm rotating-moving mechanism 171 and rotate both the couch 12 and the projector 10 to make the second straight line L2 parallel to the first straight line L1. Thus, the X-ray diagnosis apparatus of this embodiment can create X-ray images from X-rays irradiated to the subject in the same direction before and after enlargement instruction.

Incidentally, there is a case where the mechanism 17 rotates the projector 10 around the isocenter C30 after the relative movement of the couch 12 and the projector 10. In this case, a shift occurs between the position of the first isocenter C3 before enlargement instruction and the position of the second isocenter C4 after rotation. The isocenter C30 is an isocenter in a straight line connecting the X-ray focal point C5 and the center C10 of the detection surface A1 of the flat panel detector 101 after the relative movement of the couch 12 and the projector 10. For example, the calculator 151 calculates the length and direction of the shift based on the position of the first isocenter C3 before enlargement instruction, the position of the isocenter C30, and the rotation angle of the projector 10, and outputs the length and direction thus obtained to the system controller 15. The system controller 15 outputs a displacement instruction based on the length and direction obtained by the calculator 151 to the mechanism 17. The mechanism 17 then relatively moves the couch 12 and the projector 10 based on the displacement instruction. Thus, also in this case, the position of the first isocenter C3 before enlargement instruction matches the second isocenter.

### [Operation]

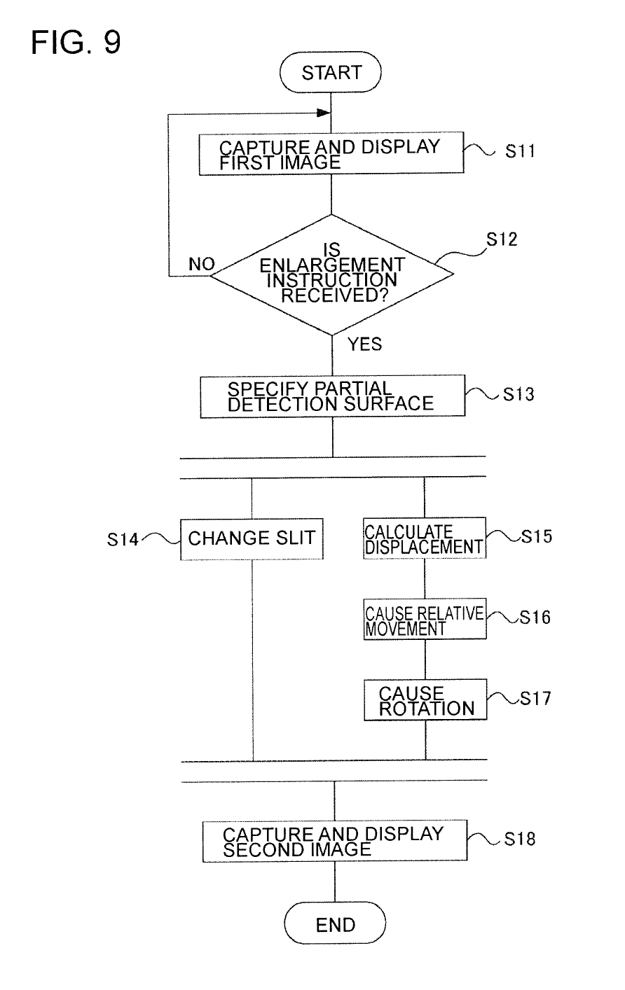

FIG. 9 is a flowchart illustrating the operation of the X-ray diagnosis apparatus of this embodiment.

### (Steps S11 to S16)

Steps S11 to S16 correspond to steps S01 to S06 in FIG. 7.

### (Step S17)

The system controller 15 outputs, to the mechanism 17, a rotation instruction representing rotational movement that makes the second straight line L2 connecting the center C2 of the partial detection surface A2 and the X-ray focal point C5 parallel to the first straight line L1 connecting the center C1 of the detection surface A1 and the X-ray focal point C5. Upon receipt of the rotation instruction, the mechanism 17 relatively rotates the couch 12 and the projector 10 to thereby make the second straight line L2 parallel to the first straight line L1.

### (Step S18)

Step S18 corresponds to step S07 in FIG. 7.

According to this embodiment, having received an enlargement instruction, the system controller 15 outputs, to the mechanism 17, a rotation instruction representing rotational movement that makes the second straight line L2 connecting the center C2 of the partial detection surface A2 and the X-ray focal point C5 parallel to the first straight line L1 connecting the center C1 of the detection surface A1 and the X-ray focal point C5. Upon receipt of the rotation instruction, the mechanism 17 relatively rotates the couch 12 and the projector 10. Thereby, the second straight line L2 becomes parallel to the first straight line L1. Thus, the X-ray diagnosis apparatus of this embodiment can create X-ray images from X-rays irradiated to the subject in the same direction before and after enlargement instruction.

### Third Embodiment

### Configuration

Described below is an X-ray diagnosis apparatus of a third embodiment. This embodiment is different from the first and the second embodiments in the configuration of the projector 10, the system controller 15, and the mechanism 17. In the following, the differences from the first and the second embodiments are mainly explained.

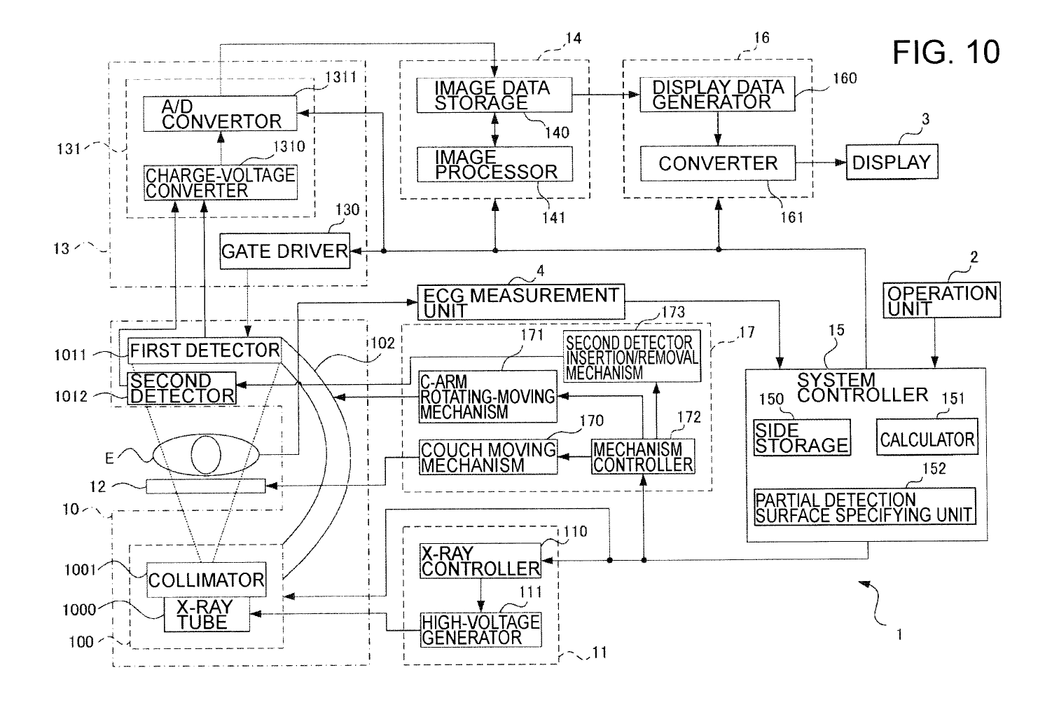

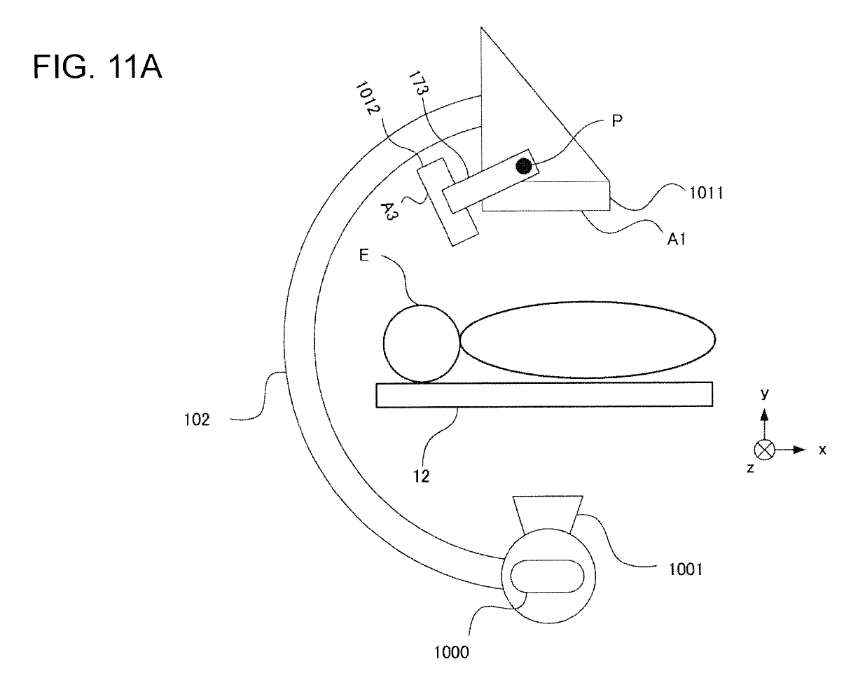

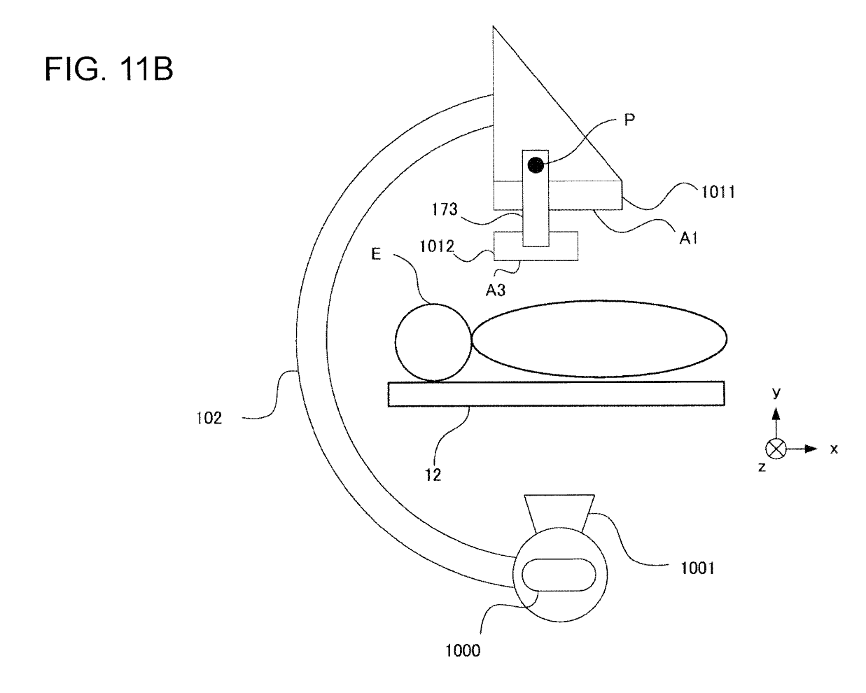

FIG. 10 is a block diagram illustrating the configuration of the X-ray diagnosis apparatus 1 of this embodiment. FIGS. 11A and 11B are schematic diagrams illustrating the outline of the projector 10 of this embodiment. The projector 10 includes a first detector 1011 and a second detector 1012. The first detector 1011 corresponds to the flat panel detector 101 of the first embodiment. The mechanism 17 includes a second detector insertion/removal mechanism 173.

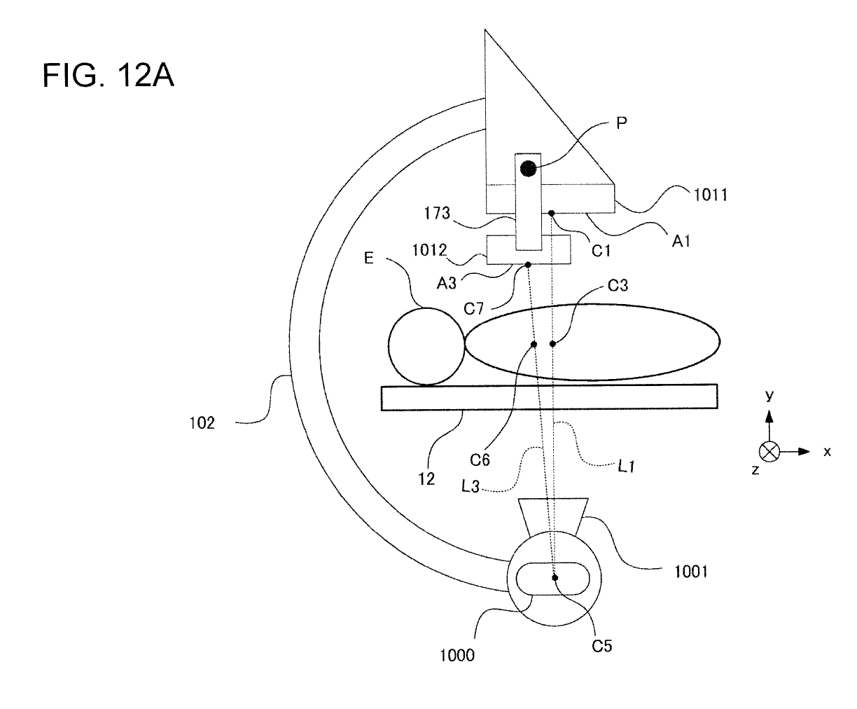

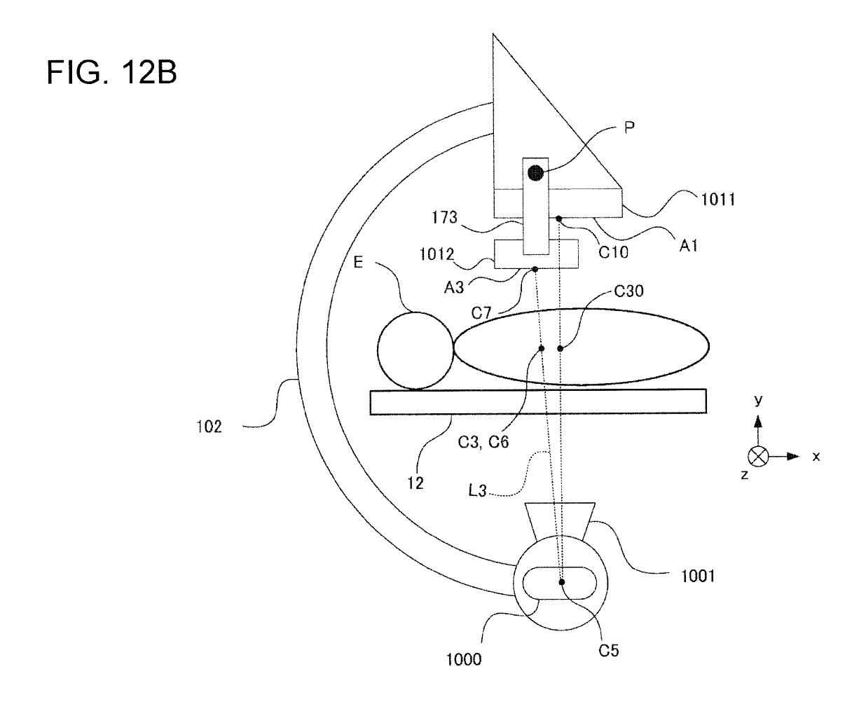

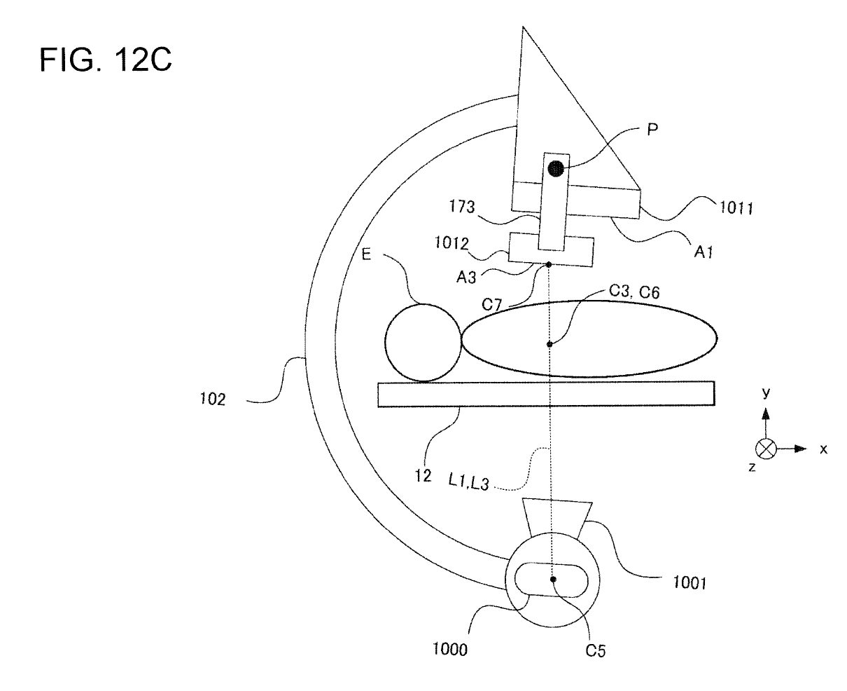

The second detector 1012 is configured to be insertable between the first detector 1011 and the subject E. For example, the second detector insertion/removal mechanism 173 inserts/removes the second detector 1012 in/from between the first detector 1011 and the subject E. FIG. 11A schematically illustrates the second detector 1012 before being inserted in between the first detector 1011 and the subject E. FIG. 11B schematically illustrates the second detector 1012 inserted between the first detector 1011 and the subject E. For example, the second detector insertion/removal mechanism 173 is formed in the shape of an arm. One end of the second detector insertion/removal mechanism 173 is rotatably attached to a predetermined position P above the first detector 1011 in the projector 10 (+y direction in FIGS. 11A and 11B). The other end of the second detector insertion/removal mechanism 173 is fixed to the second detector 1012. As the second detector insertion/removal mechanism 173 rotates around the predetermined position P, the second detector 1012 is inserted/removed in/from between the first detector 1011 and the subject E. Detection data based on X-rays detected by the second detector 1012 is output to the charge-voltage converter 1310. FIG. 12A is a schematic diagram illustrating the couch 12, the subject E, and the projector 10 before the mechanism 17 relatively moves the couch 12 and the projector 10. FIG. 12B is a schematic diagram illustrating the couch 12, the subject E, and the projector 10 after the mechanism 17 relatively moves the couch 12 and the projector 10. FIG. 12C is a schematic diagram illustrating the couch 12, the subject E, and the projector 10 after the mechanism 17 relatively rotates the couch 12 and the projector 10. A more detailed description is presented later.

The second detector 1012 is a flat panel detector having a higher spatial resolution for X-ray detection than that of the first detector 1011. In this case, an image based on X-rays detected by the second detector 1012 has a higher resolution than an image based on X-rays detected by the first detector 1011. As an example, an indirect-conversion flat panel detector is used as the first detector 1011, while a direct-conversion flat panel detector is used as the second detector 1012.

Having received an insertion instruction to insert the second detector 1012, the system controller 15 outputs, to the mechanism 17, a displacement instruction representing a direction in which the third isocenter C6 corresponding to a detection surface A3 of the second detector 1012 is brought close to the position of the first isocenter C1 corresponding to the detection surface A1 of the first detector 1011. This means that the partial detection surface A2, the center C2 of the partial detection surface A2, and the second isocenter C4 in the first embodiment are replaced by the detection surface A3 of the second detector 1012, the center C7 of the detection surface A3 of the second detector 1012, and the third isocenter C6 in the third embodiment. The position information of the detection surface A3 is specified based on the control information of the second detector insertion/removal mechanism 173. The insertion instruction is provided by the user through the operation unit to the system controller 15.

Incidentally, upon receipt of an insertion instruction to insert the second detector 1012, the system controller 15 may output, to the mechanism 17, a rotation instruction representing rotational movement that makes a third straight line L3 connecting the center C6 of the detection surface A3 of the second detector 1012 and the X-ray focal point C5 parallel to the first straight line L1 connecting the center C1 of the detection surface A1 of the first detector 1011 and the X-ray focal point C5. This means that the second straight line L2 in the second embodiment is replaced by the third straight line L3 in the third embodiment.

Having received the displacement instruction, the mechanism 17 relatively moves the couch 12 and the projector 10. This means that the partial detection surface A2, the center C2 of the partial detection surface A2, and the second isocenter C4 in the first embodiment are replaced by the detection surface A3 of the second detector 1012, the center C7 of the detection surface A3 of the second detector 1012, and the third isocenter C6 in the third embodiment.

In addition, upon receipt of the rotation instruction, the mechanism 17 relatively rotates the couch 12 and the projector 10. This means that the second straight line L2 in the second embodiment is replaced by the third straight line L3 in the third embodiment.

FIG. 12A is a schematic diagram illustrating the couch 12, the subject E, and the projector 10 before the mechanism 17 relatively moves the couch 12 and the projector 10. FIG. 12B is a schematic diagram illustrating the couch 12, the subject E, and the projector 10 after the mechanism 17 relatively moves the couch 12 and the projector 10. FIG. 12C is a schematic diagram illustrating the couch 12, the subject E, and the projector 10 after the mechanism 17 relatively rotates the couch 12 and the projector 10. For example, in X-ray fluoroscopy, after viewing an image based on the first detector 1011, the user may require enlarged display of this image by using an image based on the second detector 1012 having a higher resolution than the image. In response to the insertion instruction to insert the second detector 1012, the X-ray diagnosis apparatus 1 of the third embodiment can match the isocenter after the insertion instruction with the position of the isocenter before the insertion instruction, thereby making X-ray irradiation directions to the subject E before and after the insertion instruction parallel to each other.

Incidentally, upon receipt of an enlargement instruction for an image based on X-rays detected by the second detector 1012, the X-ray diagnosis apparatus 1 may display the image in an enlarged scale with a configuration in which the flat panel detector 101 of the first embodiment is replaced by the second detector 1012.

### [Operation]

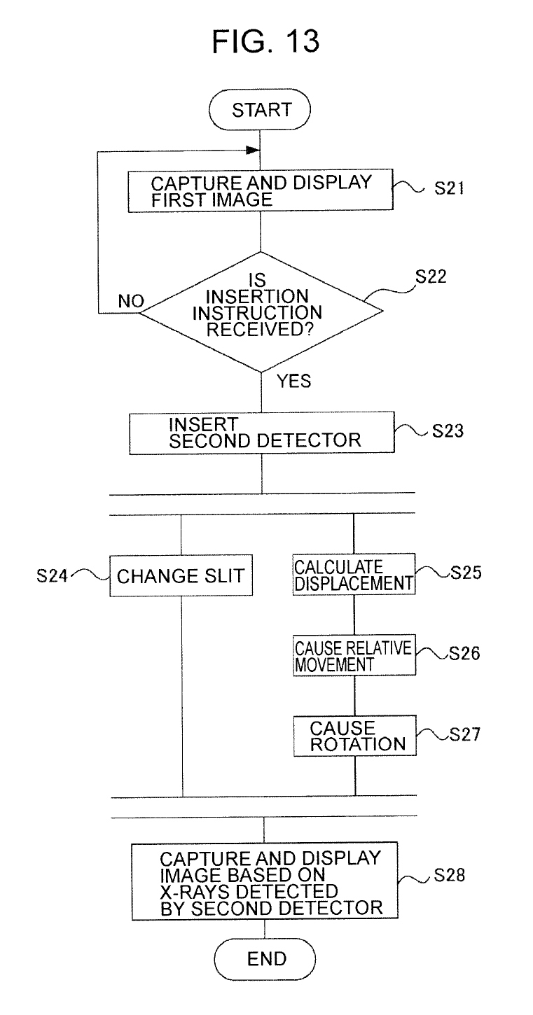

FIG. 13 is a flowchart illustrating the operation of the X-ray diagnosis apparatus 1 of this embodiment.

### (Step S21)

The X-ray diagnosis apparatus 1 captures the first image and displays it on the display 3. At this time, the X-ray generator 100 irradiates the detection surface A1 with X-rays, and the first detector 1011 detects X-rays that have passed through the subject E and are incident on the detection surface A1. The first detector 1011 outputs first detection data to the projection data generator 131. The projection data generator 131 outputs projection data based on the first detection data to the image data generator 14. The image data generator 14 generates image data based on the projection data, and outputs it to the display controller 16. The display controller 16 generates display data based on the image data, and displays it on the display 3.

### (Steps S22 and S23)

Having received an insertion instruction (YES in step S22), the system controller 15 controls the mechanism 17 to insert the second detector 1012 in between the first detector 1011 and the subject E. On the other hand, when the system controller 15 receives no insertion instruction (NO in step S22), the process returns to step S21.

### (Step S24)

The collimator 1001 changes the slit based on the detection surface A3 of the second detector 1012. Thus, X-rays are irradiated from the X-ray tube 1000 toward the detection surface A3.

### (Step S25)

The calculator 151 calculates the displacement amount of the third isocenter C6 corresponding to the detection surface A3 with respect to the first isocenter C3 corresponding to the detection surface A1 based on the detection surface A1 and the detection surface A3. The calculator 151 outputs the displacement amount thus obtained to the system controller 15. The system controller 15 outputs a displacement instruction corresponding to the displacement amount fed from the calculator 151 to the mechanism 17.

### (Step S26)

Having received the displacement instruction from the system controller 15, the mechanism 17 relatively moves the couch 12 and the projector 10 to match the position of the third isocenter C6 with the position of the first isocenter C3. Incidentally, the steps S24, S25, and S26 are in the parallel processing relationship.

### (Step S27)

The system controller 15 outputs, to the mechanism 17, a rotation instruction representing rotational movement that makes the third straight line L3 connecting the center C7 of the detection surface A3 and the X-ray focal point C5 parallel to the first straight line L1 connecting the center C1 of the detection surface A1 and the X-ray focal point C5. Having received the rotation instruction, the mechanism 17 relatively rotates the couch 12 and the projector 10 to thereby make the third straight line L3 parallel to the first straight line L1.

### (Step S28)

The X-ray diagnosis apparatus 1 captures an image based on X-rays detected by the second detector 1012 and displays it on the display 3. At this time, the X-ray generator 100 irradiates the detection surface A3 with X-rays, and the second detector 1012 detects X-rays that have passed through the subject E and are incident on the detection surface A3. The second detector 1012 outputs detection data to the projection data generator 131. The projection data generator 131 outputs projection data based on the detection data to the image data generator 14. The image data generator 14 generates image data based on the projection data, and outputs it to the display controller 16. The display controller 16 generates display data based on the image data to provide enlarged display on the display 3. With this, the operation of FIG. 13 ends.

In the X-ray diagnosis apparatus 1 of this embodiment, the projector 10 includes the first detector 1011 and the second detector 1012. Having received an insertion instruction to insert the second detector 1012, the system controller 15 outputs, to the mechanism 17, a displacement instruction representing a direction in which the third isocenter C6 corresponding to the detection surface A3 of the second detector 1012 is brought close to the position of the first isocenter C1 corresponding to the detection surface A1 of the first detector 1011. Besides, having received an insertion instruction to insert the second detector 1012, the system controller 15 outputs, to the mechanism 17, a rotation instruction representing rotational movement that makes the third straight line L3 connecting the center C6 of the detection surface A3 of the second detector 1012 and the X-ray focal point C5 parallel to the first straight line L1 connecting the center C1 of the detection surface A1 of the first detector 1011 and the X-ray focal point C5. Having received the displacement instruction, the mechanism 17 relatively moves the couch 12 and the projector 10. In addition, upon receipt of a rotation instruction, the mechanism 17 relatively rotates the couch 12 and the projector 10. In response to the insertion instruction to insert the second detector 1012, the X-ray diagnosis apparatus 1 matches the isocenter after the insertion instruction with the position of the isocenter before the insertion instruction, thereby making X-ray irradiation directions to the subject E before and after the insertion instruction parallel to each other. Thus, it is possible to match the positions of the isocenter and enable X-rays to be irradiated in parallel directions to the subject for images before and after the insertion of the second detector.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray diagnosis apparatus, comprising:
a couch on which a subject is placed;
a projector including an X-ray tube having a cathode and an anode configured to receive electrons from the cathode and irradiate X-rays to the subject, and a first detector configured to detect X-rays that have passed through the subject and are incident on a detection surface;
first circuitry configured to display a first image generated based on first detection data from the projector on a display; and
second circuitry configured to, upon receipt of an enlargement instruction to display an enlarged image of part of a site of the subject illustrated in the first image, control the first circuitry to display a second image generated based on second detection data obtained by detecting X-rays incident on a partial detection surface that is a partial area corresponding to an anode side in the detection surface as the enlarged image on the display.

2. The X-ray diagnosis apparatus of claim 1, wherein, based on an enlarged size contained in the enlargement instruction and a side corresponding to the anode side among sides of the detection surface, the partial detection surface is the partial area having at least part of the side corresponding to the anode side as a side.

3. The X-ray diagnosis apparatus of claim 1, further comprising a mechanism configured to relatively move the couch and the projector, wherein
the second circuitry is configured to, upon receipt of the enlargement instruction, output to the mechanism a displacement instruction representing a direction in which a first isocenter corresponding to the detection surface and a second isocenter corresponding to the partial detection surface are brought close to each other based on the detection surface and the partial detection surface, and
the mechanism is configured to, upon receipt of the displacement instruction, relatively move the couch and the projector.

4. The X-ray diagnosis apparatus of claim 3, wherein
the second circuitry is configured to calculate a displacement amount of the second isocenter relative to the first isocenter, and output the displacement instruction including the displacement amount to the mechanism, and
the mechanism is configured to, upon receipt of the displacement instruction, relatively move the couch and the projector based on the displacement amount such that position of the second isocenter matches position of the first isocenter.

5. The X-ray diagnosis apparatus of claim 3, wherein, upon receipt of the displacement instruction, the mechanism moves the projector.

6. The X-ray diagnosis apparatus of claim 3, wherein, upon receipt of the displacement instruction, the mechanism moves the couch.

7. The X-ray diagnosis apparatus of claim 3, wherein
the second circuitry is configured to, upon receipt of the enlargement instruction, output to the mechanism a rotation instruction representing rotational movement that makes a second straight line connecting center of the partial detection surface and an X-ray focal point parallel to a first straight line connecting center of the detection surface and the X-ray focal point, and
the mechanism is configured to, upon receipt of the rotation instruction, relatively rotate the couch and the projector.

8. The X-ray diagnosis apparatus of claim 1, further comprising a mechanism configured to relatively move the couch and the projector, wherein
the projector further includes a second detector configured to be insertable between the first detector and the subject,
the second circuitry is configured to, upon receipt of an insertion instruction to insert the second detector, output to the mechanism a displacement instruction representing a direction in which a third isocenter corresponding to a detection surface of the second detector is brought close to position of a first isocenter corresponding to the detection surface of the first detector, and
the mechanism is configured to, upon receipt of the displacement instruction, relatively move the couch and the projector.

9. The X-ray diagnosis apparatus of claim 1, further comprising a mechanism configured to relatively move the couch and the projector, wherein
the projector further includes a second detector configured to be insertable between the first detector and the subject,
the second circuitry is configured to, upon receipt of an insertion instruction to insert the second detector, output to the mechanism a rotation instruction representing rotational movement that makes a third straight line connecting center of a detection surface of the second detector and an X-ray focal point parallel to a first straight line connecting center of the detection surface of the first detector and the X-ray focal point, and
the mechanism is configured to, upon receipt of the rotation instruction, relatively rotate the couch and the projector.
